(19) United States
(12) Patent Application Publication
(10) Pub. No.: US 2023/0201767 A1
(43) Pub. Date: Jun. 29, 2023

(54) METHOD FOR DENITRATION OF FLUE GAS

(71) Applicant: BEIJING JIAOTONG UNIVERSITY, Beijing (CN)

(72) Inventors: Hong YAO, Beijing (CN); Jinyuan MA, Beijing (CN); Liang ZHANG, Beijing (CN); Sheng TIAN, Beijing (CN); Lu LU, Beijing (CN)

(73) Assignee: BEIJING JIAOTONG UNIVERSITY, Beijing (CN)

(21) Appl. No.: 17/813,432

(22) Filed: Jul. 19, 2022

(30) Foreign Application Priority Data
Dec. 29, 2021 (CN) 202111638742.3

(51) Int. Cl. B01D 53/84; B01D 53/56; C12N 1/20

(52) U.S. Cl. CPC B01D 53/84; B01D 53/56; C12N 1/20; B01D 2251/95; C02F 2101/16

(57) ABSTRACT

The disclosure belongs to the technical field of flue gas treatment and provides a method for denitration of flue gas. The method includes in the presence of anammox bacteria, subjecting a NOₓ-containing flue gas and an ammonia water to an anammox reaction.

### CROSS REFERENCE TO RELATED APPLICATION

This application claims priority from Chinese Patent Application No. 202111638742.3, filed Dec. 29, 2021, the disclosure of which is incorporated herein by reference in its entirety as part of the present application.

### FIELD

The present disclosure relates to the technical field of flue gas treatment, in particular to a method for denitration of flue gas.

### BACKGROUND

The large amount of flue gas produced from the process of industrialization is one of the main culprits of the global greenhouse effect. The main pollutants in flue gas are NOₓ (NO, NO₂, N₂O), sulfur dioxide, and dust particles.

At present, the most common methods for denitration of flue gas include selective catalytic reduction (SCR) and nonselective catalytic reduction (SNCR). The SCR method requires the use of a catalyst, temperature controlled within the range of 300 to 400° C., and the controlled amount of ammonia in the flue gas during the reaction. This method consumes a great amount of energy, involves the selection of catalyst(s), is complex in procedures, and is resource intensive.

The SNCR method does not involve the selection and use of catalyst(s), but it requires a higher temperature in the range of 850° C. to 1000° C., and a relatively high ammonia escape rate. The SNCR method involves high energy consumption, high ammonia escape rate, environmental pollution, and wastes resources.

### SUMMARY

In view of this, an object of the present disclosure is to provide a method for denitration of flue gas. In the method according to the present disclosure, an anaerobic ammonia oxidation (anammox) reaction is adopted to realize denitration of flue gas, with low energy consumption, simple procedures, and a low ammonia escape rate.

The present invention provides the following technical solutions.

Disclosed is a method for denitration of flue gas, comprising the steps of

subjecting a NOₓ-containing flue gas and an ammonia water to an anammox reaction in the presence of anammox bacteria.

In some embodiments, a molar ratio of NH₄⁺ in the ammonia water to NO in the NOₓ-containing flue gas is in the range of 0.8:1 to 1.2:1.

In some embodiments, the NOₓ-containing flue gas contains not more than 15 kg/h of SOₓ, not more than 2.2 kg/h of a particulate substance, and 14-25 kg/h of NOₓ.

In some embodiments, the ammonia water has an NH₄⁺ concentration of 200-1,000 mg/L.

In some embodiments, the anammox reaction is performed at a temperature range of 30-35° C.

In other embodiments, the anammox reaction is performed in a membrane reactor, wherein the membrane reactor comprises a shell and a plurality of membrane tubes. The membrane tubes have membrane filaments with anammox bacteria attached thereto.

In some embodiments, the anammox bacteria comprise mainly Candidatus Brocadia.

In some embodiments, the anammnox bacteria come from sludge, and the sludge has a Volatile Suspended solids (VSS)/Suspended Solid (SS) value of 0.75-0.95; the sludge is inoculated in an amount of ⅕-⅓ of the effective volume of the membrane reactor; the sludge is inoculated with the dose of at 3,000-10,000 mg. SS/L.

In some embodiments, the sludge is taken from a Sequencing Batch Reactor (SBR), and the SBR reactor has a volume loading of removal nitrogen at 0.8-1.0 kgN/m³·d.

In some embodiments, a residence time of the NO-containing flue gas in the membrane reactor is in the range of 5-10 s.

The present disclosure provides a method for denitration of flue gas, comprising the step of

in the presence of anammox bacteria, subjecting a NOₓ-containing flue gas and an ammonia water to an anammox reaction In the method according to the present disclosure, NOₓ in the flue gas is removed in the presence of anammox bacteria. Generally, in the factory areas where denitration of the flue gas is needed, wastewater containing ammonia is produced. By the method according to the present disclosure, the treatment problem of ammonia water could be solved on the spot with low energy consumption. Also, the anammox reaction could he conducted at low temperature, which reduces energy consumption. The invention results in an anammox reaction with high efficiency and low ammonia escape rate. In addition, the method according to the present disclosure is simple to n operate.

### BRIEF DESCRIPTION OF THE DRAWINGS

FIG. 1 shows a schematic diagram of the structure of a membrane reactor used in the method for denitration of flue gas according to some embodiments of the present disclosure.

FIG. 2 shows a schematic diagram of a system used in the method for denitration of flue gas according to some embodiments of the present disclosure.

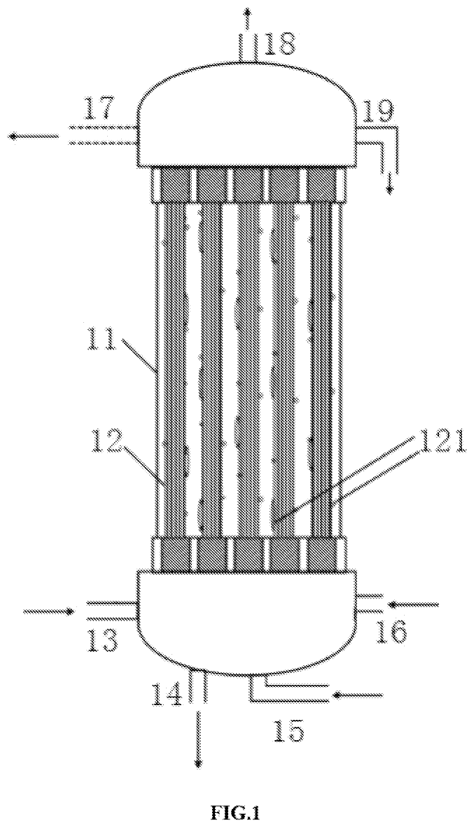

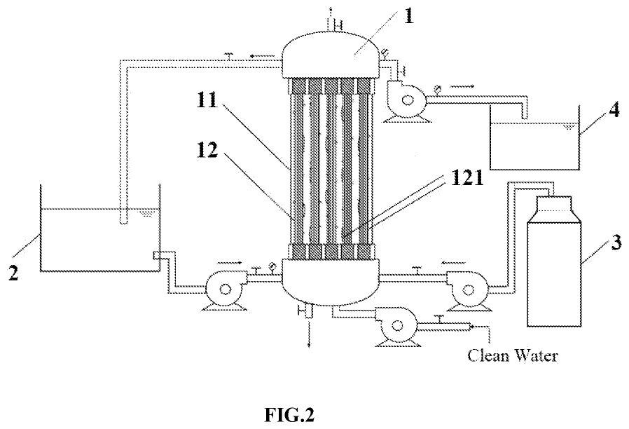

In FIGS. 1 and 2, 1 represents a membrane reactor, 11 represents a shell, 12 represents membrane tubes, 121 represents membrane filaments, 13 represents a water inlet, 14 represents a sludge outlet, 15 represents a backwash water inlet, 16 represents an air inlet, 17 represents a cross-flow port, 18 represents an air outlet, and 19 represents a water outlet; 2 represents an ammonia-water container, 3 represents a desulfurization tower, and 4 represents a discharged-water container.

### DETAILED DESCRIPTION

The present disclosure provides a method for denitration of flue gas, comprising the steps of

subjecting a NOₓ-containing flue gas and an ammonia water to an anammox reaction in the presence of anammox bacteria.

In some embodiments of the present disclosure, unless otherwise specified, the raw materials used in the present disclosure are commercially available.

In some embodiments of the present disclosure, a molar ratio of NH₄⁺ in the ammonia water to NO in the NOₓ-containing flue gas is in the range of 0.8:1 to 1.2:1.

In some embodiments of the present disclosure, the NOₓ-containing flue gas contains not more than 15 kg/h of SOₓ, not more than 2.2 kg/h of a particulate substance, and 14-25 kg/h of NOₓ. In the present disclosure, NOₓ in the NOₓ-containing flue gas comprises NO, N₂O and NO₂. In some embodiments, a mass content of NO in the NOₓ-containing flue gas is not less than 90%. In the present disclosure, the sulfide content in the NOₓ-containing flue gas is controlled to ensure the smooth progress of the anammox reaction, and to prevent an acidic pH during the anammox reaction caused by excessive sulfide content. Excessive sulfide content may result in reduced reaction efficiency. In the present disclosure, the concentration of the particulate substance in the NOₓ-containing flue gas is controlled to prolong the service life of the membrane reactor.

In some embodiments of the present disclosure, the ammonia water has an NH₄⁺ concentration of 200-1,000 mg/L.

In some embodiments of the present disclosure, the anammox reaction is performed at a temperature of 30-35° C.

In some embodiments of the present disclosure, the anammox reaction is performed in a membrane reactor. In some embodiments of the present disclosure, a schematic diagram of the structure of the membrane reactor is shown in FIG. 1. In some embodiments of the present disclosure, the membrane reactor comprises a shell 11 and a plurality of membrane tubes 12, wherein the membrane tubes 12 are provided with membrane filaments 121. In some embodiments of the present disclosure, the membrane filaments 121 have a micropore size of approximately 0.1 μm. In the present disclosure, anammox bacteria are attached to the membrane filaments.

In some embodiments of the present disclosure, the anammox bacteria comprise mainly Candidatus Brocadia.

In some embodiments of the present disclosure, the anammox bacteria come from sludge. In some embodiments of the present disclosure, the sludge has a VSS/SS value of 0.75-0.95, and preferably 0.91. In some embodiments of the present disclosure, the sludge is inoculated in an amount of ⅕-⅓ of the effective volume of the membrane reactor, and preferably ⅕. In some embodiments of the present disclosure, the sludge is inoculated with the dose of 3,000-10,000 mg SS/L, and preferably 4,000-8,000 mg SS/L.

In some embodiments of the present disclosure, the sludge is taken from a SBR reactor. In some embodiments of the present disclosure, the SBR reactor has a volume loading of removal nitrogen at 0.8-1.0 kgN/m ³ d, and preferably 0.97 kgN/m³·d.

In the present disclosure, the membrane filaments of the membrane tubes provide a good attachment carrier for anammox bacteria, and the anammox bacteria could be attached to the membrane filaments. The anammox bacteria thereon could consume ammonia wastewater and NOₓ in the flue gas, and metabolize normally. During normal metabolism, metabolites are secreted. Under the action of metabolites, anammox bacteria gradually aggregate to form large aggregates, finally forming a relatively stable biofilm with the ability to resist external shocks, which consists of anammox bacteria, and their secreted metabolites.

In some embodiments of the present disclosure, the membrane reactor is further provided with a water inlet 13, a sludge outlet 14, a backwash water inlet 15, an air inlet 16, a cross flow outlet 17, an air outlet 18, and a water outlet 19.

In some embodiments of the present disclosure, the NOx-containing flue gas is introduced into the membrane reactor 1 through the air inlet 16, and the ammonia water is introduced into the membrane reactor 1 through the water inlet 13.

In some embodiments of the present disclosure, a residence time of the NOₓ-containing flue gas in the membrane reactor is in the range of 5-10 s, and preferably 6 s.

In some embodiments of the present disclosure, the flow rate of the ammonia water is in the range of 0.1-1 m³/h.

In some embodiments of the present disclosure, the ammonia water is stored in an ammonia-water container 2 before being introduced into the membrane reactor.

In some embodiments of the present disclosure, the NOₓ-containing flue gas comes from a desulfurization tower 3.

FIG. 2 shows a schematic diagram of a system used in the method for denitration according to some embodiments of the present disclosure, in which, 1 represents a membrane reactor, 2 represents an ammonia-water container, 3 represents a desulfurization tower, and 4 represents a discharged-water container; valve(s) or pump(s) are provided on pipelines between the ammonia-water container 2 and the membrane reactor 1, between the desulfurization tower 3 and the membrane reactor 1, between the discharged-water container 4 and the membrane reactor 1, and between any two of inlets and outlets.

The method for denitration according to the present disclosure is described below in conjunction with the system.

In the system, the ammonia water in the ammonia-water container 2 is introduced into the membrane reactor 1 through the water inlet 13. When the flow rate of the ammonia water is too large, the ammonia water returns to the ammonia-water container through the cross flow port 17.

The NOₓ-containing flue gas after desulfurization in the desulfurization tower 3 is introduced into the membrane reactor 1 through the air inlet 16. In the presence of the anammox bacteria, the NOₓ-containing flue gas and the ammonia water are subjected to an anammox reaction, and N₂ is generated. The generated N₂ and other gases are overflowed through the air outlet 18 to the air or collected for further utilization.

The ammonia water treated in the membrane reactor 1 is discharged into the discharged-water container 4 through the water outlet 19.

When the membrane tubes are blocked or polluted, which adversely affects the function of the membrane reactor, clean water is introduced into the membrane reactor through the backwash water inlet 15 to rinse the membrane reactor. In some embodiments, the rinsing comprises air-water backwashing, gas backwashing or water backwashing.

The solids produced after treating in the membrane reactor 1 are discharged through the sludge outlet 14.

The method for denitration of flue gas according to the present disclosure will be described in detail below with reference to the examples. Such examples are illustrative and should not be construed as limiting the scope of the present invention.

### Example 1

An ammonia water was used, which had an NH₄⁺ concentration of 260 mg/L.

A simulated NOₓ-containing flue gas was used, which comprised 300 ppm of NO.

The ammonia water was introduced into the membrane reactor through the water inlet, and the NOₓ-containing flue gas was introduced into the membrane reactor through the air inlet. The NOₓ-containing flue gas contacted with the ammonia water in the membrane reactor and underwent an anammox reaction. A residence time of the NOₓ-containing flue gas in the membrane reactor was 6 s. A molar ratio of NH₄⁺ from the ammonia water to NO from the simulated NOₓ-containing flue gas was controlled to be in the range of 0.8:1 to 1.2:1 by controlling the flow rate of the ammonia water in the membrane reactor. The temperature in the membrane reactor was 33° C., and the anammox bacteria (mainly Candidatus Brocadia) in the membrane reactor were provided through the sludge inoculation, and the sludge was taken from a SBR reactor with volume loading of removal nitrogen at 0.97 kgN/m³·d. The sludge had a VSS/SS value of 0.91. The sludge was inoculated in an amount of ⅕ of the effective volume of the membrane reactor. The sludge was inoculated with the dose of 4,000 mgSS/L. The produced purified gas was directly discharged through the gas outlet of the membrane reactor, and the produced water was discharged through the water outlet of the membrane reactor.

After treating for 14 h, the water discharged was tested. The results are as follows: NH₄⁺ therein was reduced to 15 mg/L from 260 mg/L, which iconforms to wastewater discharge standards; the NO concentration in the purified gas was 50 ppm, which conforms to flue gas emission standards.

### Example 2

An ammonia water was used, which had an NH₄⁺ concentration of 400 mg/L.

A simulated NON-containing flue gas was used, which comprised 800 ppm of NO.

The ammonia water was introduced into the membrane reactor through the water inlet, and the NOₓ-containing flue gas was introduced into die membrane reactor through the air inlet. The NOₓ-containing flue gas contacted with the ammonia water in the membrane reactor and underwent an anammox reaction. A residence time of the NOₓ-containing flue gas in the membrane reactor was 6 s. A molar ratio of NH₄⁺ from the ammonia water to NO from the simulated NOₓ-containing flue gas was controlled to be in the range of 0.8:1 to 1.2:1 by controlling the flow rate of the ammonia water in the membrane reactor. The temperature in the membrane reactor was 33° C., and the anammox bacteria (mainly Candidatus Brocadia) in the membrane reactor were provided through sludge inoculation, and the sludge was taken from a SBR reactor with a nitrogen-removing load of 0.97 kgN/m³·d. The sludge had a VSS/SS value of 0.91. The sludge was inoculated in an amount of ⅕ of the effective volume of the membrane reactor. The sludge was inoculated with the dose of 4,000 mgSS/L. The produced purified gas was directly discharged through the gas outlet of the membrane reactor, and the produced water was discharged through the water outlet of the membrane reactor.

After treating for 14 h, the water discharged was tested. The results are as follows: NH₄⁺ therein is reduced to 20 mg/L from 400 mg/L, which conforms to wastewater discharge standards; the NO concentration in the purified gas is 70 ppm, which conforms to flue gas emission standards.

### Example 3

An ammonia water was used, which had an NH₄⁺ concentration of 400 mg/L.

A simulated. NOₓ-containing flue gas was used, which comprised 15 kg/h of SOₓ and 800 ppm of NO.

The ammonia water was introduced into the membrane reactor through the water inlet, and the NOₓ-containing flue gas was introduced into the membrane reactor through the air inlet. The NOₓ-containing flue gas contacted with the ammonia water in the membrane reactor and underwent an anammox reaction. A residence time of the NOₓ-containing flue gas in the membrane reactor was 6 s. A molar ratio of NH₄⁺ from the ammonia water to NO from the simulated NOₓ-containing flue gas was controlled to be in the range of 0.8:1 to 1.2:1 by controlling the flow rate of the ammonia water in the membrane reactor. The temperature in the membrane reactor was 33° C., and the anammox bacteria (mainly Candidatus Brocadia) in the membrane reactor were provided through the sludge inoculation, and the sludge was taken from a SBR reactor with a volume loading of removal nitrogen at 0.97 kgN/m³·d. The sludge had a VSS/SS value of 0.91. The sludge was inoculated in an amount of ⅕ of the effective volume of the membrane reactor. The sludge was inoculated with the dose of 8,000 mgSS/L. The produced purified gas was directly discharged through the gas outlet of the membrane reactor, and the produced water was discharged through the water outlet of the membrane reactor

After treating for 14 h, the water discharged was tested. The results are as follows: NH₄⁺ therein is reduced to 20 mg/L from 400 mg/L, which conforms to wastewater discharge standards; the NO concentration in the purified gas is 20 ppm, which conforms to flue gas emission standards.

The above examples represent only preferred embodiments of the present disclosure and those skilled in the art may imagine improvements and modifications falling within the scope of the present disclosure.

1. A method for denitration of flue gas, comprising the step of subjecting a NOₓ-containing flue gas and an ammonia water to an anammox reaction in the presence of anammox bacteria.

2. The method of claim 1, wherein a molar ratio of NH₄⁺ in the ammonia water to NO in the NOₓ-containing flue gas is in the range of 0.8:1 to 1.2:1.

3. The method of claim 1, wherein the NOₓ-containing flue gas contains not more than 15 kg/h of SOₓ, not more than 2.2 kg/h of a particulate substance, and 14-25 kg/h of NOₓ.

4. The method of claim 1, wherein the ammonia water has an NH₄⁺ concentration of 200-1,000 mg/L.

5. The method of claim 1, wherein the anammox reaction is performed at a temperature of 30-35° C.

6. The method of claim 1, wherein the anammox reaction is performed in a membrane reactor, and wherein the membrane reactor comprises a shell and a plurality of membrane tubes, and wherein the membrane tubes are provided with membrane filaments with anammox bacteria attached to the membrane filaments.

7. The method of claim 6, wherein the anammox bacteria comprise mainly Candidatus Brocadia.

8. The method of claim 7, wherein the anammox bacteria come from sludge, and the sludge has a VSS/SS value of 0.75-0.95;
the sludge is inoculated in an amount of ⅕-⅓ of the effective volume of the membrane reactor; and
the sludge is inoculated with the dose of 3,000-10,000 mgSS/L.

9. The method of claim 8, wherein the sludge is taken from a SBR reactor, and the SBR reactor has a volume loading of removal nitrogen at 0.8-1.0 kg/m³·d.

10. The method of claim 6, wherein a residence time of the NOₓ-containing flue gas in the membrane reactor is in the range of 5-10 s.

11. The method of claim 2, wherein the NOₓ-containing flue gas contains not more than 15 kg/h of SOₓ, not more than 2.2 kg/h of a particulate substance, and 14-25 kg/h of NOₓ.

12. The method of claim 2, wherein the ammonia water has an NH₄⁺ concentration of 200-1,000 mg/L.

13. The method of claim 5, wherein the anammox reaction is performed in a membrane reactor, and wherein the membrane reactor comprises a shell and a plurality of membrane tubes, and wherein the membrane tubes are provided with membrane filaments containing anammox bacteria attached to the membrane filaments.
